(19) United States
(12) Patent Application Publication
(10) Pub. No.: US 2008/0011613 A1
(43) Pub. Date: Jan. 17, 2008

(54) Method of electrolytically depositing a pharmaceutical coating onto a conductive osteal implant

(76) Inventor: Rizhi Wang, Delta (CA)

Correspondence Address: Gary D. E. Pierce;Pate Pierce & Baird, 215 South State Street, Suite 550, Salt Lake City, UT, 84111, US

(21) Appl. No.: 11/658,186

(22) PCT Filed: Jul. 21, 2005
(86) PCT No.: PCT/CA05/01157

§ 371 Date: Jun. 18, 2007

Related U.S. Application Data
(60) Provisional application No. 60/589,584, filed on Jul. 21, 2004.

(51) Int. Cl. C25D 9/02; A61K 9/50

(52) U.S. Cl. 205/318; 427/2.21

(57) ABSTRACT

A method of electrolytically depositing a pharmaceutical coating onto a conductive osteal implant. The implant is submerged into an electrolytic cell containing an electrolysis solution of the pharmaceutical and acts as a cathode. When current is applied to the electrolytic cell, the pharmaceutical coating forms on the implant. The pharmaceutical can comprise bisphosphonates, including calcium salts. The implants can comprise any conductive material suitable for use as an osteal implant. The implants can also be electrolytically coated with calcium phosphate before coating with a pharmaceutical.

### RELATED APPLICATIONS

This application claims the benefit of U.S. Provisional Application No. 60/589,584 filed 21 Jul. 2004.

### TECHNICAL FIELD

The invention relates to osteal implants and methods of coating osteal implants.

### BACKGROUND

Osteal implants are commonly used in surgical and dental procedures, including joint replacement procedures. Each year, hundreds of thousands of joint replacements are performed in the US and Canada. They have been established as an effective solution for those suffering from various joint diseases including arthritis, osteoporotic fractures, cancer, and avascular necrosis. One of the critical challenges with osteal implants is aseptic loosening (1, 2, 3), which can cause loosening of the implants, resulting in pain to patients and high revision costs to the health care system.

Hip replacements are one example of a commonly performed joint replacement. Although current hip replacement operations are successful in relieving pain and restoring movement, 20 percent of replaced hips fail within 20 years and will need revision surgeries.

A typical total hip replacement consists of a metallic femoral stem, a femoral head fabricated from cobalt-chromium-molybdenum alloy (CoCr) articulating against a polymeric acetabular cup fabricated from ultrahigh molecular-weight polyethylene (UHMWPE). Because CoCr is much harder than UHMWPE, the relative motion under load at the articulating surface would cause extensive wear to the polyethylene cup. The average in vivo wear rate of polyethylene cup could be as high as 0.1-0.2 mm/year, corresponding to hundreds of millions of wear debris particles released into the surrounding tissues (2, 3). The polyethylene particles generated are very small in size (˜0.5 micron) and, once they enter the so-called effective joint space, would induce inflammatory responses and periprosthetic osteolysis or bone resorption, which has been recognized as the main cause of implant failure (4).

The process of wear debris-induced osteolysis involves multiple biologic steps. The initial response to the wear particles is the phagocytosis of the particles by macrophages. The ingestion of particulate debris is associated with the release of cytokines and other mediators of inflammation. These factors then lead to osteoclast activation and bone resorption at the implant interface (2, 5, 6). Besides polyethylene, metal particles and bone cement particulates also contribute to osteolysis in the same way (6). The wear debris-associated periprosthetic osteolysis poses a long-term threat to implant longevity.

The highly stiff metallic implants could also induce another type of osteoclast-mediated bone resorption through stress-shielding. Following the hip replacement, the metallic components take the load, and the bone tissue in the proximal femur is unstressed. As a result, adaptive bone remodeling or bone resorption occurs. The stress shielding induced bone loss may affect the long-term stability as well as bone stock availability for revision surgery (7).

To improve the longevity of orthopaedic implants, attempts are underway to develop new materials and alternative bearing surfaces with reduced wear rates, including “highly crosslinked” UHMWPE, CoCr metal-on-metal systems, and ceramic on ceramic bearing systems (2, 3, 8). One concern, however, is that the reduction in debris volume is often accompanied by significant changes in their properties. For example, wear rates of metal-on-metal design can be two orders of magnitude less than those of conventional CoCr/UHMWPE design, producing metallic debris mostly less than 90 nm. However, with such small debris size, the metal-on-metal joints are expected to release about 100 times more wear particles than the conventional Co—Cr/UHMWPE counterpart (8, 9). This may raise new concerns such as ion release (10). Other attempts include reduction of femoral head size (2, 8), application of calcium phosphate coatings (11) and surface modifications (12) to promote bone apposition and limit access of wear debris to the bone-implant interface. Despite the improvements, wear is still an inevitable consequence of implantation of artificial materials for joint replacement because of the nature of joint movement. Therefore, the biochemical pathway of debris induced osteoclastic bone resorption remains unaffected and the risk of aseptic loosening persists.

Therapeutic interventions offer a promising solution to preventing aseptic loosening. Two types of drugs are being studied to inhibit the process of osteolysis. The first types of drugs are anti-inflammatory drugs that inactivate the inflammatory mediators produced by macrophages in response to wear debris (13). The second types of drugs directly inhibit excessive osteoclast function in periprosthetic bone. The most effective and promising drugs in inhibiting osteoclastic bone resorption are bisphosphonates.

Bisphosphonates, or 1,1-disphosphonates, are a unique family of drugs (e.g. etidronate, alendronate, zoledronate etc.) known for their potent ability to inhibit osteoclast activity. They have been used clinically to treat diseases of enhanced bone resorption, including Paget's disease, hypercalcemia of malignancy, and osteoporosis (14). Recent animal studies have proven that bisphosphonates have significant efficacy in inhibiting particle-induced bone resorption (15, 16, 17), peri-implant osteolysis (18), and stress-shielding induced bone loss (19).

Interestingly, enhanced peri-implant bone growth was also reported recently when bisphosphonates were immobilized on implant surfaces (20, 21, 22). Bisphosphonate treatment could result in a 115% increase in mineralized bone in acetabular implant porosities (23). One research group also confirmed in a recent paper that bisphosphonates have proliferative effects on osteoblasts and induce differentiation of osteoprogenitor cells towards the osteoblast pathway (24).

While current studies have shown promising results of bisphosphonates in inhibiting aseptic loosening and in enhancing bone formation, there are two issues to be solved to realize clinical applications. These are local delivery and controlled release. Because wear debris induced peri-implant osteolysis is a localized process that progresses with time, the best option will be to develop a local delivery system that releases bisphosphonates over a long period of time. Current oral therapy or intravenous infusion cannot be confined to the bone-implant interface and has the side effect of reducing bone turn-over rate of the whole skeleton system and other systematic effects, such as accumulative renal toxicity (14). Local drug delivery from PMMA bone cement may not have this risk, but is not applicable for cementless acetabular cups and femoral stems.

One method used by several researchers is to immerse calcium phosphate coated implants in bisphosphonate solutions (20, 25, 26, 27). By such treatments, bisphosphonates can be immobilized on hydroxyapatite surfaces, but the amount of immobilized bisphosphonate is limited and therefore, the biological effect of such surface modifications is expected to be short-term. Another challenge is to process a uniform and highly porous calcium phosphate coating so that a large surface area is available for drug adsorption. Although bisphosphonates are chemically stable, they decompose at temperatures greater than 300° C. This makes most coating techniques, e.g. thermal-spray, sol-gel, inapplicable.

Therefore, there is a need for improved methods and devices to achieve localized and prolonged release of therapeutic compounds.

### SUMMARY OF INVENTION

This invention relates to osteal implants having an electrolytically deposited pharmaceutical coating and methods of making the implants. The method comprises the steps of:
1. Preparing an electrolysis solution containing the pharmaceutical, for use in an electrolytic cell;
2. Submerging the conductive osteal implant into the electrolysis solution to act as a cathode of the electrolytic cell;
3. Submerging a second conductive electrode into the electrolysis solution to act as an anode of the electrolytic cell; and
4. Applying an electrical current to the electrolytic cell to cause formation of the pharmaceutical coating onto the osteal implant.

In alternative embodiments, the method employs a three electrode electrolysis system having a working electrode, counter electrode, and a reference electrode, wherein the osteal implant comprises the working electrode, and the second conductive electrode comprises the counter electrode.

The pharmaceutical can comprise any therapeutic compound or biopharmaceutical product which is suitable for electrolytic deposition. In some embodiments of the invention, the compound comprises a bisphosphonate. In some specific embodiments of the invention, the bisphosphonate comprises etidronate or alendronate.

The osteal implant can comprise any suitable conductive material for making osteal implants, including titanium, titanium alloys, tantalum, tantalum alloys, zirconium, zirconium alloys, stainless steels, cobalt, cobalt-containing alloys, chromium containing alloys, indium tin oxide, silicon, magnesium containing alloys, conductive polymers, or any other suitable alloys. In some embodiments of the invention, the implants comprise titanium or tantalum, including porous tantalum.

In some embodiments, the osteal implants further comprise a polymer film for sealing the pharmaceutical coating to the implant.

The invention also contemplates osteal implants comprising an electrolytically deposited biomaterial coating which is further coated with a pharmaceutical compound and methods of making such implants. In some embodiments, the method comprises electrolytically depositing a coating of calcium phosphate on an osteal implant, then further coating the implant with bisphosphonate.

### BRIEF DESCRIPTION OF DRAWINGS

In figures which illustrate various embodiments of the invention

FIG. 1 depicts the surface morphology (a) and (b), cross-section (c), (45° tilted) and EDX result (d) of the electrolytically deposited calcium etidronate coating on a titanium substrate (3h, CO₂ critical point dried).

FIG. 2 depicts the morphology (a) and EDX results (b) of a calcium etidronate reference precipitate.

FIG. 3 is a graph depicting XRD data of (top to bottom) the electrochemically deposited calcium etidronate coating (3h), calcium etidronate reference precipitate, and titanium alone.

FIG. 4 is a graph depicting FTIR data of (top to bottom): etidronic acid, reference precipitate, and electrolytically deposited coating (3h).

FIG. 5 depicts images of bare porous tantalum (Ta); a) is a photograph of a Ta plug (the cylinder is 5 mm high). b-d) are high resolution SEM micrographs of porous Ta implants provided by Zimmer, Inc.

FIG. 6 illustrates the three-electrode electrolytic deposition cell for processing a porous Ta cylinder (as a working electrode).

FIG. 7 depicts micrographs of the calcium phosphate (Ca—P) coating on a porous Ta implant. The SEM photographs are of the same location at various magnifications. Coating thickness is 2˜5 μm.

FIG. 8 is a micrograph of the surface morphology of a Ca—P coated Ta implant after soaking in alendronate solution for 7 days.

FIG. 9 depicts SEM micrographs of the morphology of a Ca-alendronate drug coating deposited on porous Ta.

FIG. 10 depicts: (left) ion chromatograms of (top to bottom): dissolved ELD coating, standard alendronate solution and deionized water. Peak 1: water of sample plug, 2: Cl⁻, 3: CO₃²⁻, 4: alendronate; (right) FTIR spectra of the ELD coating and Ca-alendronate precipitate.

FIG. 11 is a SEM micrograph of the morphology of a PLGA encapsulated Ca-alendronate coating on a porous Ta implant.

FIG. 12(a) is a photograph showing two Ta plugs that were placed in the right antero-medial tibia of a rabbit for animal studies of the implants. FIGS. 12b,c are microradiographs of the two implanted Ta plugs.

FIG. 13 is a SEM micrograph of a calcium alendronate coated Ta implant in rabbit bone. The section was made longitudinal to the Ta plug and shows extensive bone ingrowth into the porous tantalum.

FIG. 14 is a graph depicting the concentration of alendronate in a buffer solution with time. Data presented as mean ±SD (n=3).

FIG. 15 is a graph depicting the precipitation boundary of etidronate in the presence of Ca²⁺ (Ca:etidronate ratio fixed at 2:1), determined from titration of solutions containing etidronate, Ca²⁺ with NaOH; arrow head represents the ELD solution used in the experiment (etidronate 5 mM, Ca²⁺ 10 mM, pH 4.50).

FIG. 16 is a graph of the concentration of etidronate in a buffer solution over time. Points represent mean ±standard deviation (n=3).

FIG. 17 A-D depicts electrospray-ionization mass-spectrometry (ESI-MS) spectra of comparison samples. (A) 5×10⁻⁵ M etidronic acid, (B) 1:100 diluted ELD solution, (C) dissolved reference precipitate, and (D) dissolved ELD coating; all solutions in 1:1 CH₃OH/H₂O solvent and pH 1.96-2.00.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

The inventors disclose osteal implants having a pharmaceutical coating and methods of making the implants. The osteal implants can comprise any implant suitable for implantation and which are suitable for coating with a pharmaceutical coating. The implants include orthopedic and dental implants. The dental implants include root form implants, plate form implants, subperiosteal implants, or any other type of dental implant suitable for implantation in a jaw bone. The orthopedic implants include joint replacement implants, such as hip, knee, shoulder, elbow, and spine implants.

The osteal implants are made by electrolytically depositing the pharmaceutical coating onto the implants. The method of electrolytically depositing the pharmaceutical coating on the implants results in the production of even coatings of the pharmaceutical on the osteal implants. The method involves submerging the osteal implant into an electrolytic cell. The method of electrolytically depositing the pharmaceutical coating comprises the steps of:
1. Preparing an electrolysis solution containing the pharmaceutical, for use in the electrolytic cell;
2. Submerging the conductive osteal implant into the electrolysis solution to act as a cathode of the electrolytic cell;
3. Submerging a second conductive electrode into the electrolysis solution to act as an anode of the electrolytic cell; and
4. Applying an electrical current to the electrolytic cell to cause formation of the pharmaceutical coating onto the osteal implant.

In alternative embodiments, the method employs a three electrode electrolysis system having a working electrode, counter electrode, and a reference electrode. In this system, the osteal implant comprises the working electrode, the second conductive electrode comprises the counter electrode, and a standardized reference electrode, such as a calomel electrode, comprises the reference electrode.

When current passes through the electrodes, either of the following reactions occurs at the cathode or working electrode:
2H⁺+2e→H₂↑ (1)
½O₂(aq.)+H₂O+2e→2OH⁻ (2)
The detailed electrode reaction has been reviewed (31). Because of the electrode reaction, pH local to the cathode is increased due to either generation of base (OH⁻, Eq. 2) or consumption of acid (H⁺, Eq. 1). The rise of pH causes local supersaturation and precipitation of inorganic salts onto the cathode, resulting in coating formation. In the literature, this electrodeposition technique is known as electrolytic deposition (33) or electrochemical deposition (42). To avoid confusion with traditional electrochemical deposition of metals, the term electrolytic deposition (ELD) is used throughout this application.

The method of the invention contemplates the use of two electrode electrolysis systems, three electrode electrolysis systems, or other electrolysis systems. In some embodiments of the invention, the cathode comprises the osteal implant, and the anode can comprise a platinum electrode or other suitable electrode. In other embodiments of the invention, the method employs a third, reference electrode. In specific embodiments, the reference electrode can comprise a calomel electrode. Other electrolytic deposition systems known to persons skilled in the art are also contemplated.

In the method of the invention, the cathodic potential is maintained at a low voltage, which helps to avoid formation of large hydrogen bubbles and results in an even coating.

The electrolysis solution used in the method comprises a solution of ions of the pharmaceutical which is to be coated on the implant. The pH of the solution and concentration of the pharmaceutical ions in the solution is adjusted depending on the solubility parameters of the pharmaceutical.

The pharmaceutical which is coated on the implant comprises any suitable substance which provides therapeutic benefit to a patient or animal, including pharmaceutical compounds and biopharmaceutical products. In some embodiments of the invention, the pharmaceutical can comprise compounds or biopharmaceutical products which have therapeutic benefit to a patient or animal due to extended, localized release of the compound. Such pharmaceuticals include bone-growth promoting compounds, anti-osteoclastic compounds, anti-inflammatory compounds, anti-infective compounds, antibiotic compounds, antifungal compounds, anti-viral compounds, analgesic compounds, anti-cancer compounds, anti-tumour compounds and chemotherapeutic compounds. The pharmaceutical is dissolved into the electrolytic solution into which the osteal implant is submerged and forms part of the electrolytic cell. The pharmaceutical can be any compound or biopharmaceutical product suitable for electrolytic deposition.

In one embodiment of the invention, the pharmaceutical comprises a bisphosphonate, which is useful in preventing aseptic loosening of osteal implants. The bisphosphonate can comprise any bisphosphonate, including etidronate, alendronate, risedronate, ibandronate, zoledronate, tiludronate, pamidronate and clodronate. In some embodiments, the bisphosphonate comprises a calcium salt of the bisphosphonate or any other salt suitable for electrolytic deposition, including a salt of Zn²⁺, Mg²⁺, Mn²⁺, Zn²⁺, Sr²⁺, Ba²⁺, Ag²⁺, Cu²⁺, and other cations such as Fe³⁺, Zr⁴⁺, Ti⁴⁺. In some specific embodiments, the bisphosphonate comprises calcium etidronate or calcium alendronate.

Due to their relationship with pyrophosphate and phosphate, bisphosphonates have similar chemical properties. With pH increase, bisphosphonates form insoluble compounds with divalent cations like Ca²⁺, Zn²⁺, Mg²⁺, Mn²⁺, Zn²⁺, Sr²⁺, Ba²⁺, Ag²⁺, Cu²⁺, and other cations such as Fe³⁺, Zr⁴⁺, Ti⁴⁺, etc. Therefore, the technique of electrolytic deposition discussed above can be used to deposit bisphosphonate coatings onto conductive substrates.

The method of the invention can be applied to any conductive osteal implant. Such implants can comprise any conductive material suitable for use as an osteal implant, including titanium, titanium alloys, tantalum, tantalum alloys, zirconium, zirconium alloys, stainless steels, cobalt, cobalt-containing alloys, chromium containing alloys, indium tin oxide, silicon, magnesium containing alloys, conductive polymers, or any other suitable alloys. In some embodiments of the invention, the implants comprise titanium. In other embodiments of the invention, the implants comprise tantalum, including porous tantalum.

Once the pharmaceutical coating is electrolytically deposited onto the osteal implant, the implant may be further sealed with a polymer film to further slow the release of the coating once the implant is implanted. In some embodiments of the invention, the polymer film comprises a poly-lactide-co-glycolic acid. It will be appreciated by persons skilled in the art that other suitable polymer films may also be applied to the coated implants.

In another embodiment of the invention, the method of making osteal implants with a pharmaceutical coating comprises electrolytically depositing a biomaterial coating on the implant before applying the pharmaceutical coating. The biomaterial coating can comprise calcium phosphate or calcium carbonate.

The electrolytically deposited calcium phosphate coating is porous and evenly covers both the inner and outer surfaces of a porous implant surface. ELD coating calcium phosphate thus overcomes the “line-of-sight” effect associated with the commonly used plasma spray technique. One reported drawback of ELD was that the hydrogen bubbles generated at the cathode interfere with the deposition of calcium phosphate minerals onto the cathode and the coating integrity was an issue (48). Another problem was that large brushite crystals, instead of desired small OCP or hydroxyapatite crystals, often deposited (48). A technique of depositing bubble-free, fine porous OCP coating on implants with complex geometry has been developed as described below. The method comprises the steps of:
1. Preparing an electrolysis solution containing calcium ions and bisphosphate or phosphate ions for use in an electrolytic cell, wherein the calcium to phosphorus ratio is less than 1:1;
2. Submerging a conductive osteal implant into the electrolysis solution to act as a cathode of the electrolytic cell;
3. Submerging a second conductive electrode into the electrolysis solution to act as an anode of the electrolytic cell;
4. Optionally, in cases where the conductivity of the implant is poor, adding an amount of H₂O₂ to enhance current densities and minimize hydrogen bubble formation; and
5. Applying an electrical current to the electrolytic cell to cause formation of a calcium phosphate coating onto the osteal implant, wherein the cathodic potential is maintained at a low voltage to avoid formation of large hydrogen bubbles.
   A pharmaceutical compound can be further applied to the calcium phosphate coated osteal implant.

The ratio of calcium to phosphorus in the electrolysis solution is less than 1:1, and theratio can be between 1:1 and 1:10, which results in formation of a more desirable, porous coating. In some embodiments, the ratio is 1:2.

The cathodic potential (vs. a reference electrode) is maintained at a low voltage, which avoids formation of large hydrogen bubbles. The inventors have found that the potential can range between −5 V to −0.5 V to avoid excessive bubble formation, where −0.5V is the minimum voltage required to cause a reaction to occur. In some embodiments, the range is between −3 V to −1 V.

In cases where the implant to be coated has poor conductivity (for example, when the implant has an oxide layer), H₂O₂ can be added to enhance current densities in the electrolytic cell, which minimizes hydrogen bubble formation. H₂O₂ can be added to a concentration between 0.001 mol/L to 0.05 mol/L. In some embodiments, the concentration is 0.01 mol/L.

The pharmaceutical compound can be applied to the calcium phosphate coated implant by dipping the implant into a solution containing the pharmaceutical, although other methods for applying a pharmaceutical, which are known to persons skilled in the art, are also contemplated.

In one embodiment of the invention, this method is used to electrolytically deposit a calcium phosphate coating onto porous tantalum implants. In other embodiments of the invention, the implant can be dipped into a solution of a bisphosphonate to coat the calcium phosphate coated implant with bisphosphonate.

The invention also contemplates osteal implants comprising an electrolytically deposited pharmaceutical coating, an electrolytically deposited pharmaceutical coating which is sealed with a polymer film, and an electrolytically deposited calcium phosphate coating which is further coated with a pharmaceutical compound. The invention also contemplates osteal implants made according to the methods of the invention.

Detailed examples of embodiments of the invention are discussed below to further illustrate the invention.

### EXAMPLES

The following examples are intended to illustrate various embodiments of the invention, and they are not intended to limit the scope of the invention.

### Example 1

### Electrolytically Deposited Calcium Bisphosphonate on Titanium

1. Materials and Methods

a) Preparation of Electrolysis Cell

Commercially pure titanium plates (20×20×3 mm) were mechanically ground with sand paper (800 grit) and ultrasonically cleaned successively in acetone, ethanol and distilled water each for 5 minutes. The plates were etched in 1% HF acid for 5 minutes and ultrasonically cleaned again in distilled water and stored in distilled water for further use. An electrolyte solution was prepared using bisphosphonate. The bisphosphonate used in this example was etidronic acid (1-hydroxy-ethylidene-1,1-diphosphonate, Fluka, Switzerland). Their structures are shown below. 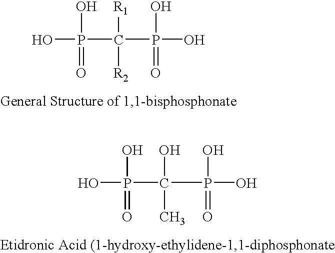
Etidronate was chosen because 1) it is being used in clinics (14); 2) studies on its calcium salts prepared in solution are available (34, 35), which makes comparisons possible; and 3) it is readily available.
b) Titration of Etidronate

Etidronic acid has a molecular weight of 206.03 (Fluka, Switzerland). The precipitation boundary of etidronate in the presence of Ca²⁺ was determined by titration and used for choosing the solution conditions suitable for electrolytic deposition (ELD). A series of 50 mL solutions containing etidronate (15, 10, 9, 5, 4, 2.5, 1.9, 1.25, 1.22, 1.15, 1.10, and 0.625 mM) and Ca(NO₃)₂ of different concentrations, with Ca:etidronate molar ratio all fixed at 2:1, were prepared and titrated with NaOH solutions until the first visual sign of turbidity (end point/the precipitation boundary). The pH and NaOH volume consumption at end points were recorded and the Ca²⁺ and etidronate concentrations at this point was calculated using equation (Eq. 3):

$$C = {C}_{0} X \frac{50}{50 + {V}_{NaOH}} \qquad ( 3 )$$

where C is the titrate (etidronate or Ca²⁺) concentration (mM) at the end point, C₀ is the titrate concentration (mM) before titration, and V_{NaOH} is NaOH volume (mL) consumption at the end point of titration.

After the precipitation boundary was established, etidronate 5.0 mM, Ca²⁺ 10 mM, and pH 4.50 were chosen as the solution conditions for ELD. The solution volume for each ELD process was set at 50 mL because the drug concentration would not be significantly decreased by coating deposition. This volume also provided reasonable space to accommodate the experiment setup, that is, two electrodes and the fixture.

c) Electrolytic Deposition

Proper amount of etidronic acid and Ca(NO₃)₂.4H₂O were weighed, dissolved and mixed to make a concentration of etidronate: 5.0 mM and Ca²⁺: 10.0 mM. The pH of the solution was adjusted to 4.36 by addition of 1M NaOH solution and monitored with a calibrated pH meter (Thermo Orion 410, Beverly, Mass., US). Electrolytic deposition was performed by two-electrode electrolysis. The cleaned titanium plates were used as cathodes and a platinum plate as the anode, separated by 5 mm. The voltage was kept constant at 2.45V (GW laboratory DC power supply GPS 1830D, Goodwill, Taiwan). After 1 to 9 hours (typically 3 hours) of deposition, a thin calcium etidronate coating formed on the cathode surface. The coated titanium plates were then rinsed repeatedly with distilled water and absolute ethanol followed by air-drying or CO₂ critical point drying.

d) Preparation of Reference Precipitate

Calcium etidronate precipitate was prepared as a reference precipitate as it is not commercially available. NaOH (1M) was added dropwise into the same solution as used in electrolytic deposition to the final pH of 5.55 to induce precipitation. Precipitation occurred immediately upon adding NaOH. The suspension was stored overnight, filtered (Whatman 44, Maidstone, UK), rinsed repeatedly with distilled water, and dried at 65° C. Because the molecular structure of etidronate has been well known to be stable under these common precipitation conditions (11), the reference precipitate thus prepared can be used as a comparison to examine whether molecular structure of etidronate and its calcium salt are altered in the coatings prepared by the ELD process.

e) Morphology and Crystallinity of Coated Samples

Samples were sputtered with Au-40% Pd alloy and characterized with scanning electron microscope (SEM, Hitachi 3000N) for surface morphology at 5 kV. Coating compositions were analyzed on un-sputtered samples by energy dispersive X-ray analysis (EDX) attached to the SEM. Both coatings and reference precipitate were characterized by X-ray diffraction (XRD, Rigaku Rotaflex RU-200BH, Cu K_{α}, 50 kV, 100 mA, 0.5°/min.). Calcium etidronate reference powder was mounted on a titanium sample holder, which also acted as an internal standard.

f) Molecular Structure Analysis

Electrospray-ionization mass-spectrometry (ESI-MS) was performed to examine whether the molecular structure of etidronate was preserved in the coating. To examine the ELD coating, the drug-coated Ti plate was immersed in 50-mL HCl solution (pH 2.00, solvent: methanol/water, V:V=1:1, methanol: Fisher, HPLC grade min. 99.9%) to completely dissolve the coating. The solution was readjusted to pH 2.00 with 1 M HCl. Methanol/water was used as the solvent to be compatible with the electrospray ionization.

Three solutions were prepared and used for comparisons:
1. 5×10⁻⁵ M etidronic acid: 20.6 mg etidronic acid was weighed and dissolved in 50 mL water; 0.5 mL was pipetted and combined with 20 mL methanol/water solution (V:V=1:1, pH adjusted at 2.00 with 1 M HCl); the final pH was 1.99.
2. 1:100 diluted ELD solution: 200 μL of the ELD solution was pipetted and combined with 20 mL methanol/water solution (conditions same as 1); pH was adjusted to 1.96 with 1 M HCl.
3. 12 mg reference precipitate was weighed and dissolved in 50-mL diluted HCl solution, 2 mL was pipetted and combined with 8 mL H₂O, and 10 mL methanol; pH was adjusted to 2.00 with 1 M HCl. The precipitate dissolved completely.

The etidronate concentrations in the above comparison solutions were similar to each other (˜5.0×10⁻⁵ M). The etidronate concentration in the solution prepared from ELD coating (comparison sample 3) was estimated to be higher. However, taking into account the fact that the coating may contain a certain amount of water, this protocol was intentionally used to prevent generating too low signals in ESI-MS. All solutions were analyzed with an electrospray-ionization mass-spectrometer operating in negative ion mode (Bruker Esquire˜LC, scan rage: m/z 60-400). To prevent intersample contamination, the spectrometer was flushed with methanol before scanning each sample until the signals from the preceding sample disappeared.

Fourier transform infrared spectroscopy (FTIR, PerkinElmer 2000, resolution mode: 1 cm⁻¹) was also used for structural examination. The coating, reference precipitate, and the etidronic acid were pressed with KBr into pellets and tested. FTIR spectrum of commercially pure hydroxyapatite [Ca₁₀(OH)₂(PO₃)₆, HAp, Fisher Scientific] was also obtained by this procedure and used for comparison.

g) In Vitro Release

To study the in vitro release properties of the electrolytically deposited etidronate coating, 15 coated samples were each placed into a conical flask containing 50 mL “physiological buffer” solution (140 mM NaCl, 1.5 mM Ca²⁺, 50 mM Tris, pH 7.40), sealed and kept at 37° C. in a water bath (40). The buffer solution has similar pH and Ca²⁺ concentrations with human plasma and has been used to study etidronate release kinetics from its Ca salt (40). Three samples were taken out after 1, 2, 4, 6, and 8 days, respectively. The solutions were quickly filtrated (to remove possible solid Ca-etidronate particles in solution, pipetted, and kept for testing. Filtration was also performed within the water bath, covered with a lid, to prevent potential re-precipitation caused by temperature differences.

Etidronate concentrations released into the solution were determined by total phosphorus analysis per ASTM D6501-99 (Standard Test Method for Phosphonate in Brines), and all chemicals used were ACS reagent grade. In brief, etidronate in solution was converted to phosphate by potassium persulfate (K₂S₂O₈) digestion in an autoclave (15 psig, 120° C., 30 min) and reacted with ammonium molybdate [(NH₄)₆Mo₇O₂₄] to form a phosphomolybdate complex. The complex was extracted with an organic phase (cyclohexane/methyl-isobutylketone, V:V=1:1) and measured calorimetrically (725 nm, Cary 50 UV-Vis spectrophotometer, Varian). The calibration curve was established by measuring standard phosphate solutions and linearity (r²=0.997) was obtained over the concentration range of 1-5×10⁻⁵M phosphate. For in vitro release measurements, solutions were pretested and sample volumes were accordingly chosen to meet the linear range. Three samples were tested for each time point.

2. Results

a) Etidronate Precipitation and ELD Solution

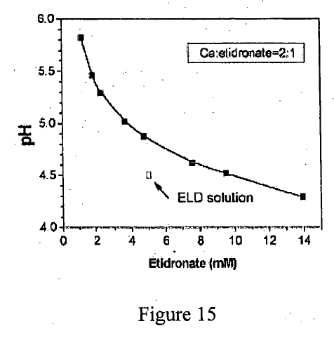

FIG. 15 depicts the precipitation boundary of etidronate in the presence of Ca²⁺, with Ca:etidronate molar ratio fixed at 2:1. The precipitation pH increased with decreasing Ca²⁺ and etidronate concentration and the increase became sharp when etidronate <2 mM. When etidronate <1.25 mM, the end point (precipitation) became less obvious and eventually no precipitation could be visually judged when etidronate <1.15 mM. Therefore, at lower concentrations, small difference in etidronate or Ca²⁺ concentration will cause a relatively high change in pH at which the precipitation can occur. This would adversely affect the robustness of ELD coating processing. The curve slope gradually decreased with increasing concentration; however, higher etidronate concentration represents higher cost and lower yield, defined as the ratio of amount of etidronate deposited/etidronic acid dissolved. As a result, an intermediate concentration of 5 mM etidronate and 10 mM Ca²⁺ was chosen as the solution for ELD. The solution pH was determined to be 4.50 because the solution at this pH was close to the precipitation boundary and found stable for a reasonably long time (>7 days) without spontaneous precipitation (ELD solution condition, see arrow head in FIG. 15).

b) Electrolytic Deposition of Etidronate

Etidronic acid dissociated into etidronate anions (Eq. 4) when pH local to the cathode rose (Eqs. 1 and 2); this increased the supersaturation over its calcium salt. When critical supersaturation was reached (i.e., the solution went up across the precipitation boundary in FIG. 15), precipitation occurred local to the Ti cathode (Eqs. 5 and 6, where H₄L denotes etidronic acid H₈C₂P₂O₇, M denotes a metal) and aggregated to form a continuous film, that is, the ELD coating.
H₄L→H₃L⁻+H⁺→H₂L²⁻+2H⁺→HL³⁻3H⁺→L⁴⁻+4H⁺ (4)
H₂L²⁻+M²⁺→MH₂L↓ (5)
L⁴⁻+2M²⁺→M₂L↓ (6).
c) Morphology and Crystallinity

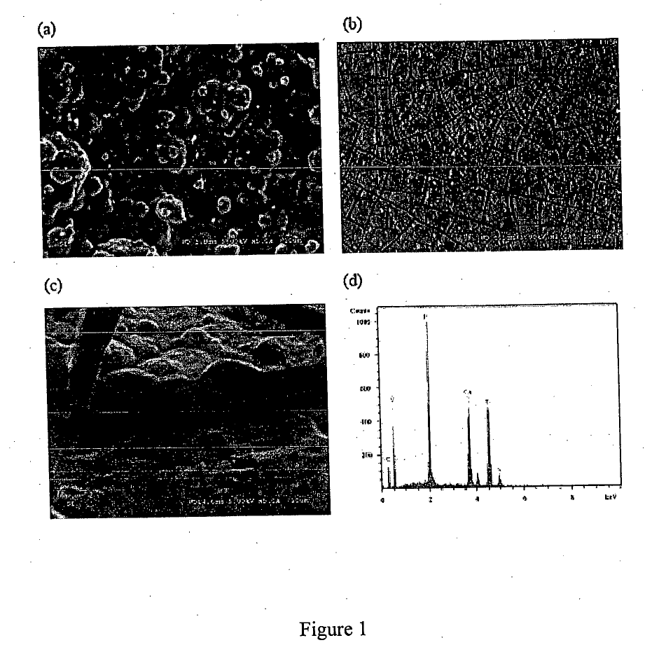

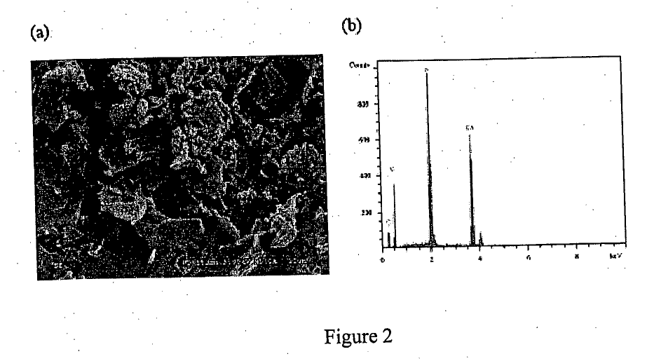

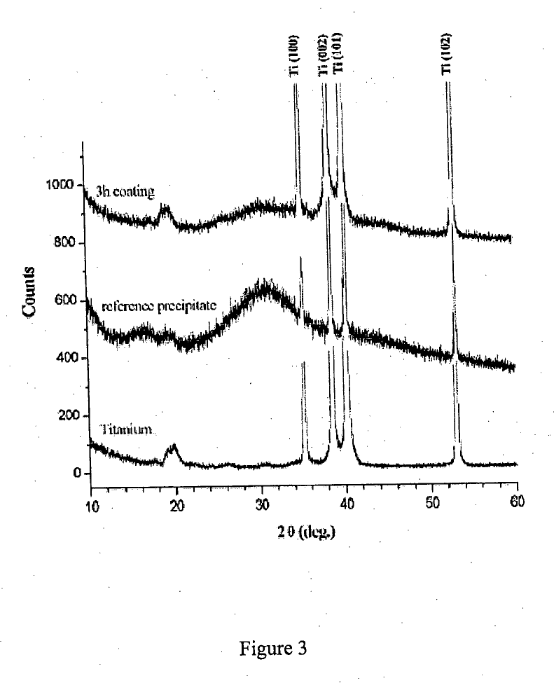

After 1 h of electrolysis, gray coatings fully covered cathode Ti substrate immersed in ELD solution. The coating color changed to white after a deposition time of 3 h, indicating growing thickness with time. The coating deposition was reproducible in all experiments performed (n>30). FIG. 1 shows the typical morphology of coating deposited at 3 h. It can be seen [FIG. 1(a)] that globules about 0.5 μm in size aggregated into larger globular domains, which further packed into a solid film. No appreciable porosity was observed. Cracks were observed at lower magnifications indicating significant shrinkage during drying [FIG. 1(b)]. The coating thickness was measured to be ˜3.7 μm at 3 h [FIG. 1(c)]. The Ca/P ratio of the coating was determined to be 0.78 by EDX [FIG. 1(d)]. As a comparison, the reference precipitate consisted of flakes and shreds of different shapes and sizes [FIG. 2(a)], and the Ca/P ratio was determined to be 1.02 by EDX [FIG. 2(b)]. XRD results (FIG. 3) of both reference precipitate and coating showed a diffuse peak at around 31°, indicating that they are amorphous. The diffraction peaks at 35.1, 38.5, 40.2, and 53.0° are from titanium substrates. The peak at 20° appeared in bare titanium has been frequently observed in the inventors' previous works, and may be due to some unknown diffractions; however, this peak appeared on all samples and would not interfere with characterizations.

d) Molecular Structure

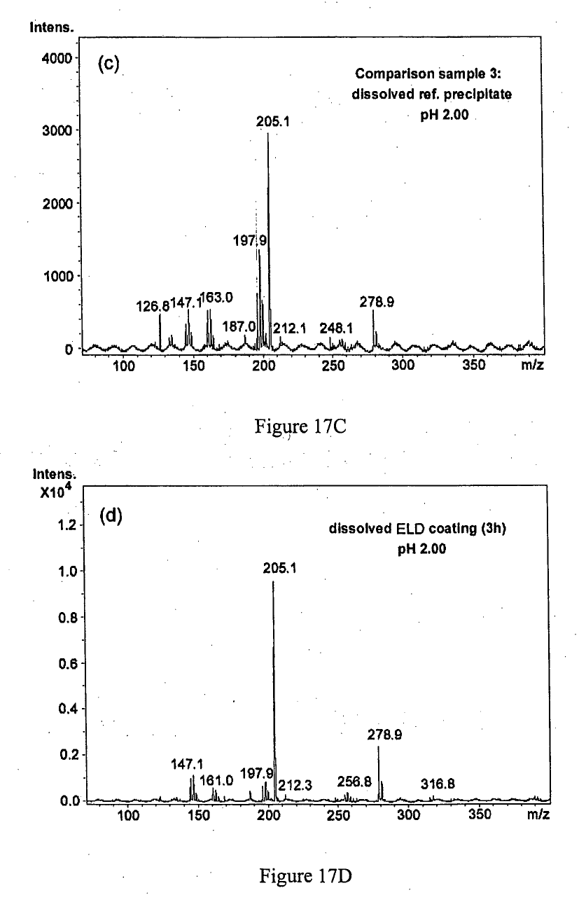

Electrospray-ionization mass-spectrometry (ESI-MS) results for the coating and the three comparison samples are shown in FIG. 17. In all spectra, the main peak appeared at the mass/charge (m/z) ratio of 205.1. This peak matches the negative ion of etidronic acid [H₃L]⁻ (m/z: 205.02), which arises from dissociation of etidronic acid (Eq. 7).
H₄L(206.03)→H₃L⁻(205.02)+H⁺ (7)
where H₄L denotes etidronic acid (H₈C₂P₂O₇), a tetrabasic acid.

Other weaker peaks may have complex origins like solvent impurities, etidronate complexation products, or adducts with other molecules/ions. Peaks with m/z around 197.9 and 161 were observed in all samples, and the peaks around 147.1 were observed for the ELD solution, reference precipitate, and the coating. No significant new peaks appeared for the coating itself. Comparison of solutions 1, 2, and 3 showed similar etidronate concentrations, and both the main and the weaker peaks had comparable intensities. In the dissolved coating, which had higher etidronate concentration, the main peak intensity was relatively higher.

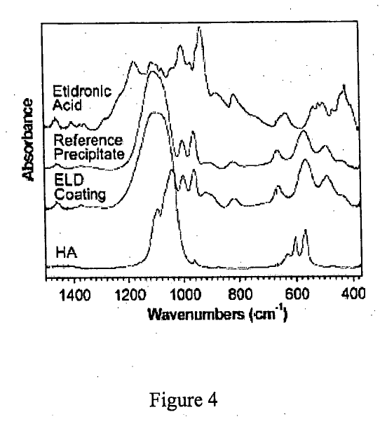

Additional structure information was obtained from FTIR, as shown in FIG. 4. Etidronic acid showed strong and broad bands at 900-1300 cm⁻¹, characteristic of P—O stretching modes, and two bands at 422 and ˜515 cm⁻¹, characteristic of bending mode of phosphonic acid (36). The coating and reference precipitate showed nearly identical IR profiles: (1) P—O stretching bands appeared at 1100, 1000, and 961 cm⁻¹; (2) the absorption at 915˜1020 cm⁻¹ were significantly reduced compared to the etidronic acid, due to decrease of the P—OH stretching mode (909˜1040 cm⁻¹) in this region (36) with proton being replaced by Ca²⁺; (3) bending mode appeared at 567 and 490 cm⁻¹.

Commercially standard hydroxyapatite was also tested for comparison. The hydroxyapatite showed stretching (1093, 1042, and 963 cm⁻¹), bending (603 and 566 cm⁻¹) of the phosphate group, and stretching (631 cm⁻¹) of the OH group. Positions and intensities of these absorbance modes were clearly different from the reference precipitate and the drug coating.

One of the concerns in ELD processing was whether the chemical structure of etidronate drugs could be kept intact during deposition and this was investigated by different characterization techniques. The coating and the reference precipitate showed similar XRD diffuse patterns at ˜2θ 31° (FIG. 3), in agreement with results reported by Browning et al. (34). Due to their amorphous nature, no structural identification could be made; however, ESI-MS clearly detected the etidronic acid (H₃L⁻ form) as the main peak in both the coating and all comparison samples. This H₃L⁻ form is also consistent with ESI-MS analyses of bisphosphonates reported in literature (43, 44). In addition, no new peaks of appreciable intensity were created by the ELD process. Although there have been no reports on the FTIR results of calcium etidronate in the literature, the FTIR profile of the ELD coating was also identical to that of the reference precipitate; therefore, the ELD coating can be confirmed as calcium etidronate.

f) In Vitro Release

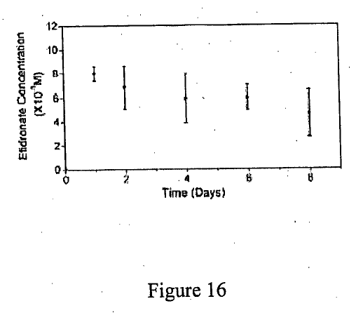

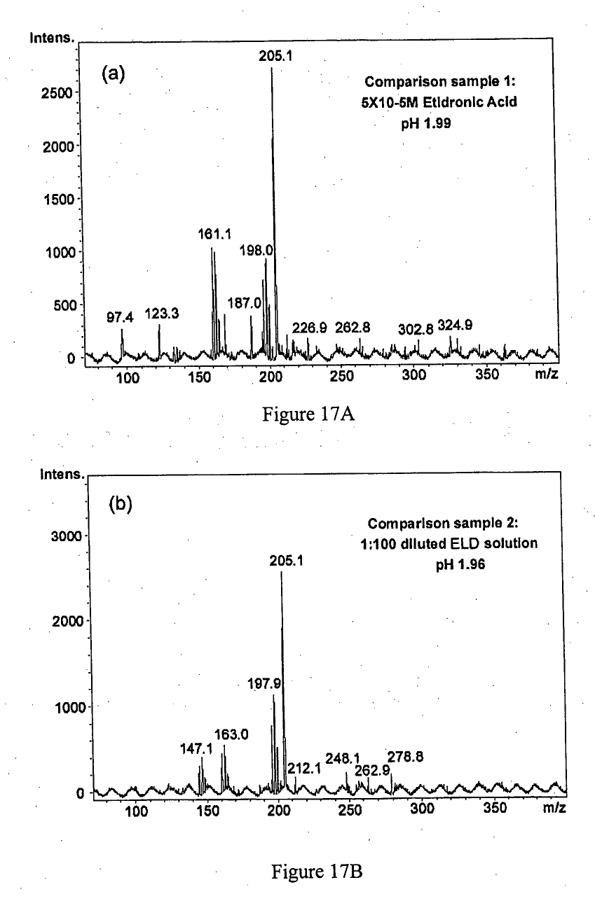

Etidronate concentrations in the buffer solution with soaking times are shown in FIG. 16. The concentration at day 1 was 8×10⁻⁵ M, and it slightly declined and remained relatively stable at ˜6×10⁻⁵ M up to day 8. No significant morphological change was observed in the first 2 days; however, coating dissolution became observable at day 4, and significant at day 8. From day 4, some solid particles formed in the solution and on the Ti substrate. For concentration measurements, these particles were removed by filtration because the target of the analyses was the free etidronate concentration, which is actually experienced by the surrounding tissue/cells. In all experiments, no macroscopic coating spallation was ever observed.

### Example 2

### Electrolytically Deposited Calcium Bisphosphonate on Porous Tantalum

1. Materials and Methods

a) Porous Tantalum Implants

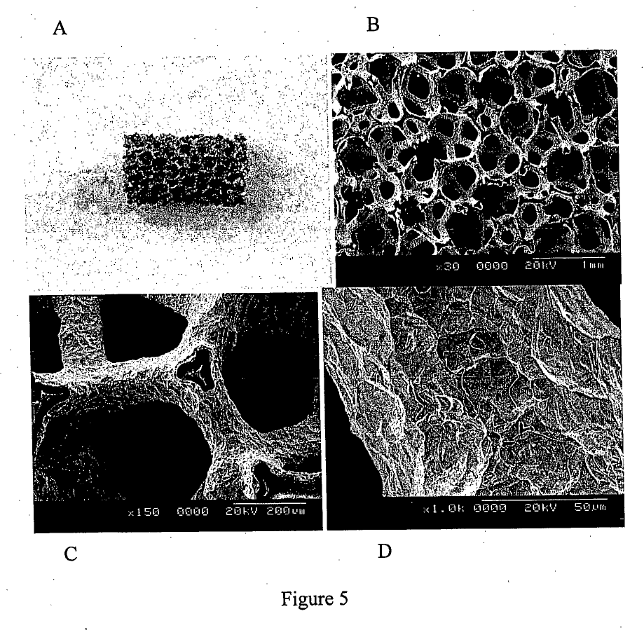

100 cylindrical porous tantalum plugs (3.15 mm in diameter and 5 mm long) were provided by Zimmer, Inc. They were sealed and sterilized with gamma rays before delivery. FIG. 5 shows optical and SEM images of the porous Ta implants at various magnifications. The porosity is estimated to be 80%.

b) Bisphosphonate and Other Materials

Alendronate (4-amino-1-hydroxybutylidene-1,1-bisphosphonate), one type of bisphosphonate, was chosen because of its high efficacy and popularity. Type I soluble collagen was used in this example. The inclusion of collagen in alendronate drug coating helps to improve mechanical integrity and bone ingrowth. Poly-(lactic-co-glycolic-acid) (PLGA, Commercial Name: Lactel, Lot #: D96056), was purchased from Birmingham Polymers, Birmingham, Ala. The lactic acid/glycolic acid ratio was 85:15.

c) Electrolytic Deposition of Coatings

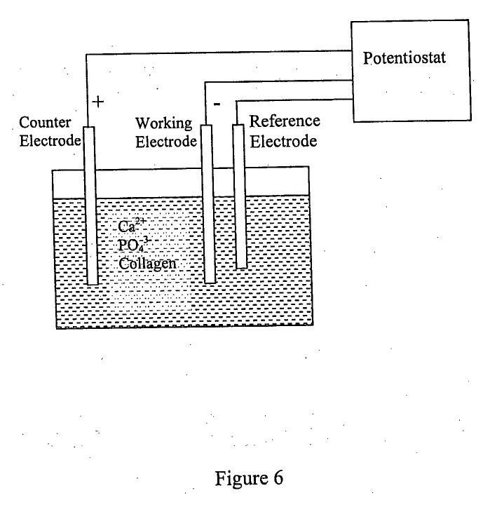

Both calcium phosphate coating and calcium alendronate coating on porous Ta were processed by electrolytic deposition. In this study, electrolytic deposition was typically carried out in a three-electrode electrochemistry system (FIG. 6) controlled by a potentiostat (Gamry PCI4/300, Gamry Instruments, Warminster, Pa.). The working electrode, i.e. cathode, was porous Ta. A platinum plate (25×25 mm) was used as the anode and a saturated calomel electrode (SCE, Aldrich) as the reference electrode.

As discussed in Example 1, the coating was realized as a result of electrochemical reaction at the cathode. When current passes through the electrodes in an electrochemical cell that contains acidic solution of the calcium salt to be deposited (e.g. calcium phosphate), either of the following reactions occurs at the cathode.
2H⁺+2e→H2(g) (Eq. 8)
½O₂(aq.)+H₂O+2e→2OH⁻ (Eq. 9)

Because of the electrode reaction, pH local to the cathode is increased due to either generation of base (OH⁻, Eq. 9) or consumption of acid (H⁺, Eq. 8). This pH rise causes local supersaturation and precipitation of inorganic salts onto the cathode (either calcium phosphate or calcium alendronate, depending on the electrolyte used). This electrolytic deposition has advantages over other coating techniques. It does not have the “line-of-sight” drawback associated with plasma spraying, and thus can make a homogenous coating on such a porous structure as porous Ta. Compared with biomimetic coating (of calcium phosphate), electrolytic deposition is faster, and does not have the spherical agglomerates found in biomimetic coatings.

d) Calcium Phosphate Coating on Ta (Ta/Ca—P)

In preparing the electrolyte for depositing calcium phosphate, the proper amounts of Ca(NO₃)₂ and NH₄H₂PO₄ were dissolved in distilled water to make 5.25 mM [Ca²⁺] and 10.5 mM [PO₄³⁻]. The pH was adjusted to 5.19-5.33 by adding 1M NaOH solution and monitoring with a calibrated pH meter (Thermo Orion 410, Beverly, Mass., US). The deposition was done using a two-electrode system (without the reference electrode shown in FIG. 6). The voltage was kept constant at 2.50V. The coating process was carried out at room temperature (20° C.) for 3 hours.

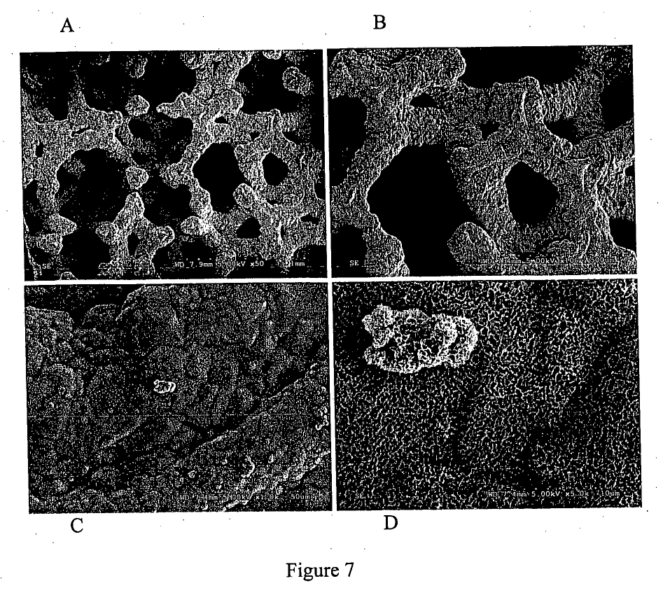

Typical calcium phosphate coating on porous Ta is shown in FIG. 7. The coating is 2-5 μm thick and is porous at high magnification with an average pore size of ˜0.5-1 μm. EDS and FTIR analysis confirmed that the coating was mainly octacalcium phosphate (OCP) with a Ca/P ratio of 1.3-1.4. The OCP coating covers the porous Ta beam uniformly without cracking. Because of the thin coating, the morphology of individual Ta crystals could still be resolved.

e) Bisphosphonate Soaked Calcium Phosphate Coating (Ta/Ca—P/BPP)

To absorb bisphosphonate drug onto OCP coated Ta implants, each of the OCP coated porous Ta samples (3.4) was soaked in a 2 ml phosphate buffer solution (PBS, pH 7.4) that contained 10⁻⁴ M/L sodium alendronate. After 7 days at 35° C., the specimens were rinsed in deionized water and air-dried.

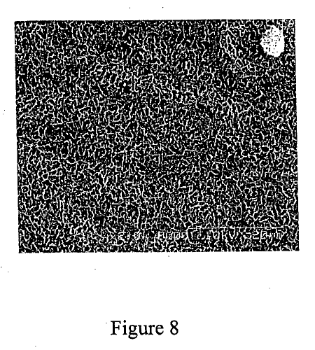

No dramatic change was observed to the porous calcium phosphate coating after alendronate soaking. However, there seems to be a very thin smear layer covering the surface (FIG. 8). HPLC tests have measured a total of ˜12.5 μg of alendronate absorbed on each Ta implant.

f) Calcium Bisphosphonate Drug Coating (Ta/CaBPP)

According to a study on precipitation boundary, the solution condition for electrolytic deposition was chosen as: alendronate 3.5×10⁻³ mol/L, Ca²⁺ 7.0×10⁻³ mol/L, pH 4.8 and solution volume 50 ml. Electrolytic deposition (ELD) was carried out in three-electrode mode. A constant cathodic potential of −1.48 V (vs SCE) was applied on the Ta. Deposition time ranged from 10 min to 60 min. After deposition, the drug-coated Ta was removed, rinsed repeatedly with distilled water and air-dried.

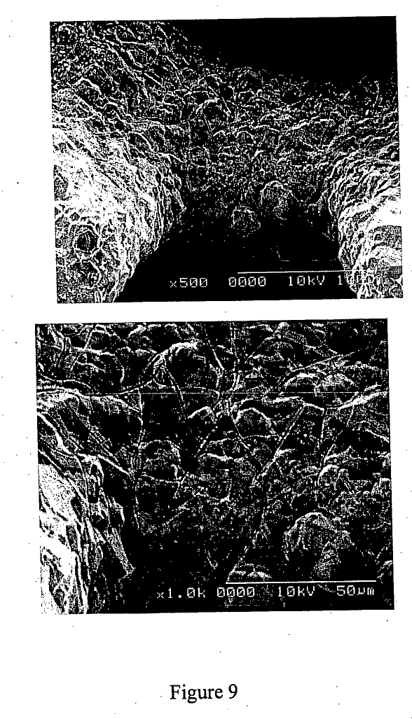

A uniform alendronate coating was obtained on both flat Ta plate and porous Ta (FIG. 9). The coating is built up of densely agglomerated spherical particles with diameter ranging from submicron to microns. Fine cracks were observed.

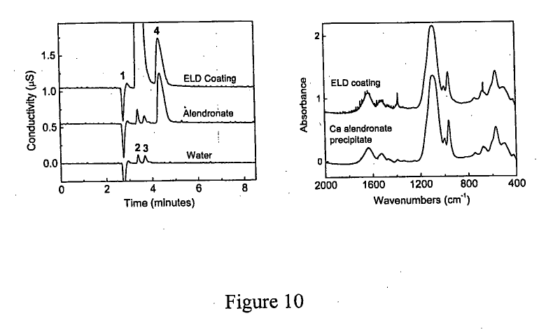

Ion chromatography and FTIR tests confirmed that the ELD drug coating was alendronate (FIG. 10). Two peaks appeared on the chromatogram of the dissolved ELD coating: a strong peak at the retention time of 3.3 minutes (peak 2) and a peak of intermediate intensity (peak 4) at 4.33 minutes (FIG. 10, left). The peak at 4.33 minutes also appeared in the chromatograms of standard alendronate solution. By testing standard solutions, the peaks at 3.3 minutes were found to be chloride. Chloride was introduced from the HCl used to dissolve the coating. The negative peak (peak 1) on all chromatograms was the sample solvent (water), which had lower conductivity than the eluent. It can be concluded from the ion chromatography tests that the anion in the ELD coating is alendronate. FTIR spectrum of the ELD coating matched that of the Ca-alendronate precipitate we prepared (FIG. 10, right). A strong absorption band at 1090 cm⁻¹ with two weaker bands at 1000 and 963 cm⁻¹ were characteristic of P—O stretching modes of phosphonate salts; the bands at 502 and 564 cm⁻¹ were characteristic of the bending absorptions of phosphonate. The great similarity between the coating and the precipitate indicated that the ELD coating was a calcium alendronate coating.

g) PLGA Sealed Calcium Bisphosphonate Drug Coating (Ta/Ca—BPP/PLGA)

An appropriate amount of PLGA was weighed and dissolved in dichloromethane (Alfa Aesar, HPLC grade: >99.7%) to make a 5% (w/w) solution. Substrates coated with Ca-alendronate were dipped into the solution, slowly withdrawn and transferred into a fridge to allow slow evaporation and drying. This was repeated 3 times for each sample.

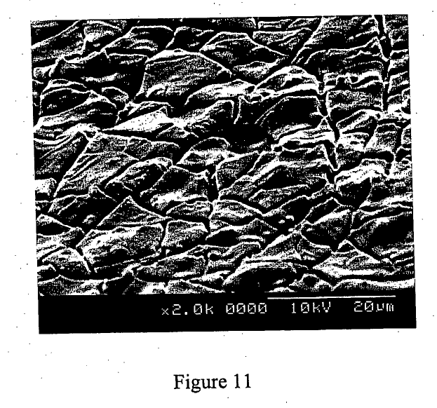

Under SEM, a thin layer of PLGA sealed the cracked calcium alendronate drug coating (FIG. 11).

h) In Vitro Drug Release

To study the in vitro release property of the Ca-alendronate coating, 18 coated samples were each placed into a sealed polyethylene vial containing 5 ml “physiological buffer” solution (0.14 mol/L NaCl, 1.5×10−3 mol/L Ca²⁺, 0.050 mol/L Tris, pH 7.4) and kept at 37° C. in a water bath. The buffer solution has similar pH and Ca²⁺ concentrations to human plasma. Three samples were taken out after every 24 hours and the solutions were measured by ion chromatography. Standard alendronate solutions were tested to establish a calibration curve and linearity (R2>0.98) was obtained over the range of 4−15×10⁻⁵ mol/L. Solutions were properly diluted to meet the linear range.

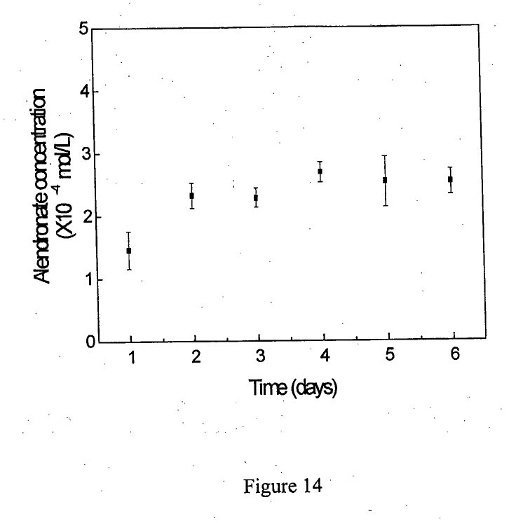

Release of alendronate from the calcium alendronate coating is shown in FIG. 14. The concentration at day 1 was 1.5×10⁻⁴ mol/L; at day 2 it reached 2.5×10⁻⁴ mol/L and kept relatively stable up to day 6. Correspondingly, no significant further coating dissolution was observed beyond day 2. In all samples, coatings survived 6 day soaking without complete dissolution.

i) Histomorphometry

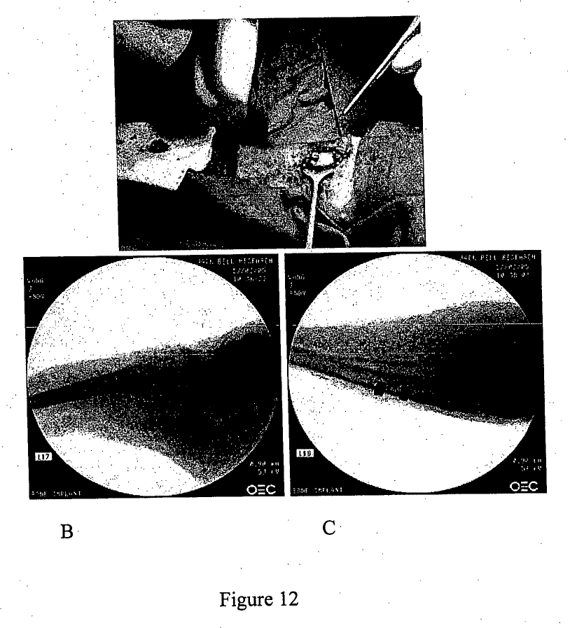

Animal studies were carried out on New Zealand White female rabbits to evaluate the biocompatibility of coatings and their effect on early bone ingrowth and fixation of implants. The implants were sterilized with β-rays before surgery. Implants were placed into the right antero-medial tibia (20 mm away from the proximal end, and 10 mm away from each other) of each rabbit (FIG. 12). The rabbits were sacrificed at week 8.

Implants were fixed together with the bone, embedded, sliced transversely to the implant, and stained (with SafarinO, Toluidine Blue, and Light Green) following standard procedures. Quantitative histomorphometric evaluations include the amount of bone per area around and inside the implant, and the length of direct bone attachment at the implant/bone interface. The polished thin sections were also analyzed with a scanning electron microscope under backscattered electron mode to study the bone mineral density and bone ingrowth.

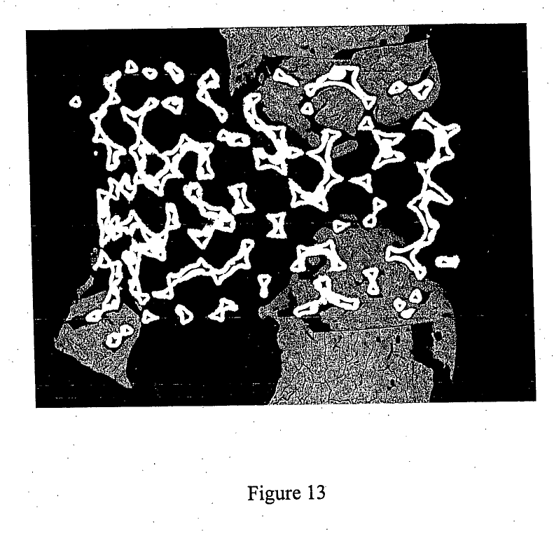

FIG. 13 is a scanning electron micrograph of a Ta implant coated with calcium alendronate in rabbit bone. The section was made longitudinal to the Ta plug. This picture shows extensive bone ingrowth into the porous tantalum. The photograph also indicates that the bisphosphonate coating is biocompatible and does not delay early bone growth.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

### REFERENCES

1. W. J. Maloney, et al. Fixation, polyethylene wear, and pelvic osteolysis in primary total hip replacement. Clin. Orthop. Rel. Res. 369 (1999) 157-164.
2. National Institute of Health. Total hip replacement consensus statement 1994. Bethesda. Md2. US. 1994, September; 12(5): 1-31.
3. Implant Wear in Total Joint Replacement: Clinical and Biologic Issues, Material and Design Considerations. Edited by Timothy M. Wright and Stuart B. Goodman. American Academy of Orthopaedic Surgeons, 2001.
4. W. H. Harris. The problem is osteolysis. Clin. Orthop. Rel. Res. 311 (1995) 46-53.
5. T. P. Schmalzried, et. al. The mechanism of loosening of cemented acetabular components in total hip arthroplasty. Clin. Orthop. 274 (1992) 60-78.
6. J. J. Jacobs, et al. Osteolysis: basic science. Clin. Orthop. Rel. Res. 393 (2001) 71-77.
7. C. A. Engh and J. D. Bobyn. The influence of stem size and extent of porous coating on femoral bone resorption after primary cementless hip arthroplasty. Clin. Orthop., 231 (1988) 7-28.
8. O. K. Muratoglu and S. M. Kurtz. Alternate bearing surfaces in hip replacement In: Sinha R K editor. Hip replacements: current trends and controversies. New York: Marcel Dekker, 2002. p. 1-46.
9. P. J. Firkins P J, et al. Quantitative analysis of wear and wear debris from metal-on-metal hip prostheses tested in a physiological hip joint simulator. Biomed. Mater. Eng. 11 (2001) 143-157.
10.a: J. J. Jacobs, et al. Cobalt and chromium concentrations in patients with metal on metal hip replacements. Clin. Orthop. 329 (1996) S256-S263. b: L. Savarino, et al. Ion release in stable hip arthroplasties using metal-on-metal articulating surfaces: a comparison between short- and medium-term results. J. Biomed. Mater. Res. 66 (2003) 450-456.
11. W. L. Jaffe and D. F. Scott. Total hip arthroplasty with hydroxyapatite-coated prostheses. J. Bone Joint Surg. 78A (1996) 1918-1934.
12. S, Nishiguchi, et al. Titanium metals form direct bonding to bone after alkali and heat treatments. Biomaterials. 22 (2001) 2525-33.
13.a: E. M. Schwarz, et al. Quantitative small-animal surrogate to evaluate drug efficacy in preventing wear debris-induced osteolysis. J. Orthop. Res. 18 (2000) 849-855. b: G. Im, et al. Osteolysis in total hip arthroplasty: biological and clinical aspects. In: R. K. Sinha editor. Hip replacements: current trends and controversies. New York: Marcel Dekker, 2002. p. 177-212.
14. H. Fleisch. Bisphosphonates in bone disease: from the laboratory to the patient. 4th edition. San Diego: Academic Press, 2000.
15. Sabokbar, et al. Bisphosphonates in bone cement inhibit PMMA particle induced bone resorption. Ann. Rheum. Dis. 57 (1998) 614-618.
16. E. M. Schwarz, et al. Quantitative small-animal surrogate to evaluate drug efficacy in preventing wear debris-induced osteolysis. J. Orthop. Res. 18 (2000) 849-855.
17. S. M. Horowitz, S. A. Algan, M. A. Purdon. Pharmacologic inhibition of particulate-induced bone resorption. J. Biomed. Mater. Res. 31 (1996) 91-96.
18. R. S. Shanbhag, C. T. Hasselman, H. E. Rubash. Inhibition of wear debris mediated osteolysis in a canine total hip arthroplasty model. Clin. Orthop. 344 (1997) 33-43.
19. D. G. Little, et al. Zoledronate acid prevents osteopenia and increases bone strength in a rabbit model of distraction osteogenesis. J. Bone Miner. Res. 18 (2003) 1300-1307.
20. S. J. Meraw and C. M. Reeve. Qualitative analysis of peripheral peri-implant bone and influence of alendronate sodium on early bone regeneration. J. Peridontol. 70 (1999) 1228-1233.
21. M. Yoshinari, et al. Bone response to calcium phosphate-coated and bisphosphonate-immobilized titanium implants. Biomaterials. 23 (2002) 2879-2885.
22. P. Tengvall, B. Skoglund, A. Askendal, P. Aspenberg. Surface immobilized bisphosphonate improves stainless-steel screw fixation in rats. Biomaterials. 25 (2004) 2133-2138.
23. A. S. Shanbhag, D. May, C. Chan, C. T. Hasselman, H. E. Rubash. Enhancing net bone formation in canine total hip components with bisphosphonates. Trans. Orthop. Res. Soc. 44 (1999) 255.
24. Gun-Il Im, S. A. Qureshi, J. Kenney, H. E. Rubash, A. S. Shanbhag, Osteoblast proliferation and maturation by bisphosphonates, Biomaterials, 25 (2004) 4105-4115.
25. Ganguli, et al. The interaction of bisphosphonates in solution and as coatings on hydroxyapatite with osteoblasts. J. Mater. Sci.: Mater. Med. 13 (2002) 923-931.
26. H. Denissen, et al. Normal osteoconduction and repair in and around submerged highly bisphosphonate-complexed hydroxyapatite implants in rat tibiae. J. Peridontol. 71 (2000) 272-278.
27. M. Yoshinari, et al. Bone response to calcium phosphate-coated and bisphosphonate immobilized titanium implants. Biomaterials 23 (2002) 2879-2885.
28. R. Wang, K. Duan, Y. W. Fan. Electrochemically deposited calcium bisphosphonate on metallic substrate as a potential anti-osteolysis coating. U.S. Provisional Patent Application No. 60/589,584.
29. D. Ostovic and G. S. Brenner. Development of subcutaneous and intramuscular formulations of calcium alendronate salts. Drug Dev. Ind. Pharm. 21 (1995) 1157-1169.
30. T. Osterman, et al. Slow-release clodronate in prevention of inflammation and bone loss associated with adjuvant arthritis. J. Pharmacol. Exp. Ther. 280 (1997) 1001-1007.
31. J. Redepenning and J. P. Mcisaac. Electrocrystallization of brushite coatings on prosthetic alloys. Chem. Mater. 2 (1990) 625-627.
32. M. Shirkhanzadeh. Bioactive calcium-phosphate coatings prepared by electrodeposition. J. Mater. Sci. Letter 10 (1991) 1415-1417.
33. Zhitomirsky. Cathodic electrodeposition of ceramic and organoceramic materials. Fundamental aspects. Adv. Colloid. Interface Sci. 97 (2002) 279-317.
34. Browning F H, Fogler H S. Effect of precipitating conditions on the formation of calcium-HEDP precipitates. Langmuir 1996; 12: 5231-5238.
35. Browning F H, Fogler H S. Effect of synthesis parameters on the properties of calcium phosphonate precipitates. Langmuir 1995; 11: 4143-4152.
36. Thomas L C. Interpretation of the infrared spectra of organophosphorous compounds. London: Heyden, 1974.
37. Lin-Vien D, Colthup N B, Fateley W G, Grasselli J G, editors. Infrared and raman characteristic frequencies of organic molecules. San Diego: Academic Press, 1991.
38. Shagidullin R R, Pudovic A N, editors. Atlas of IR spectra of organophosphorous compounds: Interpreted spectrograms. Nauka & Kluwer, Boston, 1990.
39. Liggett S J. Determination of ethane-1-hydroxy-1,1-diphosphonic acid (EHDP) in human feces and urine. Biochem Med. 1973; 7:68-77.
40. Johnston T P, Schoen F J, Levy R J. Prevention of calcification of bioprosthetic heart valve leaflets by Ca2+ diphosphonate pretreatment. J Pharmaceut Sci 1988; 77:740-744.
41. Zhang J M, Lin C J, Feng Z D, Tian Z W. Mechanistic studies of electrodeposition for bioceramic coatings of calcium phosphates by an in situ ph-microsensor techniques. J Electron Chem 1998; 452:235-240.
42. Kuo M C, Yen S K. The process of electrochemical deposited hydroxyaptite coatings on biomedical titanium at room temperature. Mater Sci Eng C 2002; 20:153-150.
43. Qing X Z, Tsai E W, Sakuma T. Pharmaceutical application of gas chromatography-mass spectrometry. 2. Ion chromatography ion-spray mass-spectrometric characterization of alendronate. J Chromatogr A 1994; 686:204-212.
44. Huikko K, Kostiainen R. Analysis of bisphosphonates by capillary electrophoresis-electrospray ionization mass spectrometry. J Chromatogr A 2000; 872:289-298.
45. Zaki M T, Ismail M I, Rizkalla E N. Metal chelates of phosphonate-containing ligands: VII. Analytical applications of 1-hydroxyethane-1,1-diphosphonic acid. Microchem J 1984; 30; 6-11.
46. Garcia-Moreno C, Serrano S, Nacher M, Farre M, Diez A, Marinoso M L, Carbonell J. Mellivobsky L, Nogues X, Ballester J, Aubia J. Effect of alendronate on cultured normal human osteoblasts. Bone 1998; 22:233-239.
47. Reinholz G G, Getz B, Pederson L, Sanders E S, Subramanian M, Ingle J N, Spelsberg T C. Bisphosphonates directly regulate cell proliferation, differentiation and gene expression in human osteoblasts. Cancer Res 2000; 60:6001-6007.
48. M. Kumar, H. Dasarathy, C. Riley, Electrodeposition of brushite coatings and their transformation to hydroxyapatite in aqueous solutions. J Biomed Mater Res 1999; 45:302-310.

1-17. (canceled)

18. A method of electrolytically depositing a bisphosphonate coating onto a conductive osteal implant, the method comprising the steps of:
a) preparing an electrolysis solution containing a bisphosphonate for use in an electrolytic cell;
b) submerging the conductive osteal implant into the electrolysis solution to act as a cathode of the electrolytic cell;
c) submerging a second conductive electrode into the electrolysis solution to act as an anode of the electrolytic cell; and
d) applying an electrical current to the electrolytic cell to cause formation of the bisphosphonate coating onto the osteal implant.

19-23. (canceled)

24. A method according to claim 18, wherein the osteal implant comprises a material selected from the group consisting of titanium, titanium alloys, tantalum, tantalum alloys, zirconium, zirconium alloys, stainless steels, cobalt, cobalt-containing alloys, chromium containing alloys, indium tin oxide, silicon, magnesium containing alloys, and conductive polymers.

25-30. (canceled)

31. A method of depositing a calcium phosphate coating onto a conductive osteal implant, the method comprising the steps of:
a) preparing an electrolysis solution containing calcium ions and bisphosphate or phosphate ions for use in an electrolytic cell, wherein the calcium to phosphorus ratio is less than 1:1;
b) submerging a conductive osteal implant into the electrolysis solution to act as a cathode of the electrolytic cell;
c) submerging a second conductive electrode into the electrolysis solution to act as an anode of the electrolytic cell;
d) optionally adding an amount of H₂O₂ to enhance current densities and minimize hydrogen bubble formation; and
e) applying an electrical current to the electrolytic cell to cause formation of a calcium phosphate coating onto an osteal implant, wherein a cathodic potential is maintained at a low voltage to avoid formation of large hydrogen bubbles.

32. A method according to claim 31, further comprising the step of applying a pharmaceutical compound onto the calcium phosphate coated osteal implant.

33. A method according to claim 31, wherein the ratio of calcium to phosphorus in the electrolysis solution is between 1:1 and 1:10.

34-36. (canceled)

37. A method according to claim 31, wherein the H₂O₂ is added t a concentration between 0.001 mol/L and 0.04 mol/L.

38. (canceled)

39. A method according to claim 32, wherein the pharmaceutical compound is selected from the group consisting of bone-growth promoting compounds, anti-osteoclastic compounds, anti-inflammatory compounds, anti-infective compounds, antibiotic compounds, antifungal compounds, anti-viral compounds, analgesic compounds, anti-cancer compounds, anti-tumor compounds and chemotherapeutic compounds.

40. (canceled)

41. A method according to claim 39, wherein the pharmaceutical compound comprises a bisphosphonate.

42. (canceled)

43. A method according to claim 41, wherein the bisphosphonate comprises calcium etidronate.

44. A method according to claim 41, wherein the bisphosphonate comprises calcium alendronate.

45. A method according to claim 31, wherein the osteal implant comprises a material selected from the group consisting of titanium, titanium alloys, tantalum, tantalum alloys, zirconium, zirconium alloys, stainless steels, cobalt, cobalt-containing alloys, chromium containing alloys, indium tin oxide, silicon, magnesium containing alloys, and conductive polymers.

46. (canceled)

47. A method according to claim 45, wherein the material is tantalum.

48-50. (canceled)

51. A method of depositing a bisphosphonate coating onto a conductive osteal implant, the method comprising the steps of:
a) preparing an electrolysis solution containing calcium ions and bisphosphate or phosphate ions for use in an electrolytic cell, wherein the calcium to phosphorus ratio is less than 1:1;
b) submerging a conductive osteal implant into the electrolysis solution to act as a cathode of the electrolytic cell;
c) submerging a second conductive electrode into the electrolysis solution to act as an anode of the electrolytic cell;
d) optionally adding an amount of H₂O₂ to enhance current densities and minimize hydrogen bubble formation;
e) applying an electrical current to the electrolytic cell to cause formation of a calcium phosphate coating onto the osteal implant, wherein a cathodic potential is maintained at a low voltage to avoid formation of large hydrogen bubbles; and
f) applying a bisphosphonate coating onto the calcium phosphate coated implant.

52-56. (canceled)

57. An osteal implant made according to the method of claim 31.

58. (canceled)

59. An osteal implant made according to the method of claim 51.

60-65. (canceled)

66. A method according to claim 51, wherein the osteal implant comprises a material selected from the group consisting of titanium, titanium alloys, tantalum, tantalum alloys, zirconium, zirconium alloys, stainless steels, cobalt, cobalt-containing alloys, chromium containing alloys, indium tin oxide, silicon, magnesium containing alloys, and conductive polymers.

67. A method according to claim 32, wherein the pharmaceutical compound is selected from the group consisting of bone-growth promoting compounds, anti-osteoclastic compounds, anti=inflammatory compounds, anti-infective compounds, antibiotic compounds, antifungal compounds, anti-viral compounds, analgesic compounds, anti-cancer compounds, anti-tumor compounds and chemotherapeutic compounds.

68. A method according to claim 32, wherein the osteal implant comprises a material selected from the group consisting of titanium, titanium alloys, tantalum, tantalum alloys, zirconium, zirconium alloys, stainless steels, cobalt, cobalt containing alloys, chromium containing alloys, indium tin oxide, silicon, magnesium containing alloys, and conductive polymers.

69. An osteal implant made according to the method of claim 32.
